(19) United States
(12) Patent Application Publication
(10) Pub. No.: US 2024/0339219 A1
(43) Pub. Date: Oct. 10, 2024

(54) Method For Measuring Trunk Balance, Method For Determining Color Of Lens For Eyewear, Method For Determining Color Of Contact Lens, Method For Determining Color Of Frame For Eyewear, And Method For Determining Color Of Wearable Item For Human

(71) Applicant: Innochi Inc., Oita (JP)

(72) Inventor: Takashi HAITANI, Awaji-shi Hyogo (JP)

(73) Assignee: Innochi Inc., Oita (JP)

(21) Appl. No.: 18/575,503

(22) PCT Filed: Dec. 20, 2022
(86) PCT No.: PCT/JP2022/046821

§ 371 Date: Dec. 29, 2023

(30) Foreign Application Priority Data
Jan. 12, 2022 (JP) 2022-002858

(51) Int. Cl. G16H 50/30; A61B 5/00; A61B 5/103; A61B 5/11; G02C 5/00; G02C 7/02; G02C 7/04

(52) U.S. Cl. CPC G16H 50/30; A61B 5/1034; A61B 5/1036; A61B 5/1116; A61B 5/4023; A61B 5/4884; A61B 5/702; G02C 7/027; G02C 5/008; G02C 7/04

(57) ABSTRACT

A more suitable eyewear lens color is selected. An eyewear lens color determination method has: a color acquisition step (step S1, step S2) of causing a person under evaluation of which trunk balance is to be measured to stand such that his/her posture becomes readily unstable upon external application of a force to the person under evaluation, then having the person under evaluation hold each of colored paper sheets of different colors in sequence, and measuring trunk balance of the person under evaluation based on a movement of the person under evaluation produced by externally applying a force thereto in order to acquire color information on the thing providing the result of good trunk balance; and a color determination step (step S3, step S4) of determining an eyewear lens color based on the color information acquired in the color acquisition step.

### TECHNICAL FIELD

The present invention relates to a technology to measure trunk balance and its applied technology.

### BACKGROUND ART

There is a conventional method to select an eyewear lens color (e.g., PTL 1).

### CITATION LIST

### Patent Literature

Patent literature 1: Japanese Patent No. 6556967

### SUMMARY OF INVENTION

### Technical Problem

It is an object of the present invention to provide a much more suitable measurement of human trunk balance for a person under evaluation, and a much more suitable determination of an eyewear lens color, a contact lens color, an eyewear frame color or a color of human-wearable thing for a person under evaluation.

### Solution to Problem

To address the above issues, a first aspect of the present invention provides a trunk balance measurement method for measuring trunk balance of a person. The trunk balance measurement method includes an external force input step and a measurement step, the external force input step being of causing a person under evaluation of which trunk balance is to be measured to have readily an unstable posture upon external application of a force to the person under evaluation, then causing the person under evaluation to hold each of things of different colors in sequence, and externally applying a force to the person under evaluation, the measurement step being of measuring trunk balance of the person under evaluation based on a movement of the person under evaluation in the external force input step. In a second aspect of the present invention, preferably, in the external force input step, the person under evaluation may be stood or sat on measuring means capable of causing the person under evaluation to have readily an unstable posture upon external application of a force to the person under evaluation.

In a third aspect of the present invention, preferably, in the external force input step, the person under evaluation may be stood on measuring means capable of causing the person under evaluation to stand to have readily an unstable posture upon external application of a force to the person under evaluation, and the measuring means may have a shape facilitating the person under evaluation having an unstable posture.

In a fourth aspect of the present invention, preferably, the measuring means may have a shape allowing a foot of the person under evaluation to be placed thereon, the shape extending from a heel to toes of the foot and having a smaller width than that of a sole of the foot, and in the external force input step, the person under evaluation may place one of his/her feet on the measuring means to stand on the one foot thereon, and then a force may be externally applied to the person under evaluation. In a fifth aspect of the present invention, preferably, the things of different colors may be colored paper sheets.

To address the above issues, a sixth aspect of the present invention provides an eyewear lens color determination method for determining an eyewear lens color using the trunk balance measurement method according to any one of the first to fifth aspects of the present invention. The eyewear lens color determination method includes: a color acquisition step of acquiring color information on the thing providing measurement results of good trunk balance in the measurement step; and a color determination step of determining an eyewear lens color based on the color information acquired in the color acquisition step.

In a seventh aspect of the present invention, preferably, in the color determination step, a plurality of candidates for eyewear lens colors may be selected based on the color information acquired in the color acquisition step, then the person under evaluation may be caused to have readily an unstable posture upon external application of a force to the person under evaluation, then the person under evaluation may be caused to wear eyewear with each eyewear lens color of the plurality of candidates in sequence, in order to measure trunk balance of the person under evaluation based on a movement of the person under evaluation produced by externally applying a force to the person under evaluation, and an eyewear lens color that provides measurement results of good trunk balance may be determined.

To address the above issues, an eighth aspect of the present invention provides a contact lens color determination method for determining a contact lens color using the trunk balance measurement method according to any one of the first to fifth aspects of the present invention. The contact lens color determination method includes: a color acquisition step of acquiring color information on the thing providing measurement results of good trunk balance in the measurement step; and a color determination step of determining a contact lens color based on the color information acquired in the color acquisition step.

In a ninth aspect of the present invention, preferably, in the color determination step, a plurality of candidates for contact lens colors may be selected based on the color information acquired in the color acquisition step, then the person under evaluation may be caused to have readily an unstable posture upon external application of a force to the person under evaluation, then the person under evaluation is caused to wear a contact lens or contact lenses with each color of the plurality of candidates in sequence, in order to measure trunk balance of the person under evaluation based on a movement of the person under evaluation produced by externally applying a force to the person under evaluation, and a contact lens color providing a measurement result of good trunk balance may be determined.

To address the above issues, a tenth aspect of the present invention provides an eyewear frame color determination method for determining an eyewear frame color using the trunk balance measurement method according to any one of the first to fifth aspects of the present invention. The eyewear frame color determination method includes: a color acquisition step of acquiring color information on the thing providing measurement results of good trunk balance in the measurement step; and a color determination step of determining an eyewear frame color based on the color information acquired in the color acquisition step.

In an eleventh aspect of the present invention, preferably, in the color determination step, a plurality of candidates for eyewear frame colors may be selected based on the color information acquired in the color acquisition step, then the person under evaluation may be caused to have readily an unstable posture upon external application of a force to the person under evaluation, then the person under evaluation may be caused to wear eyewear with each eyewear frame color of the plurality of candidates in sequence, in order to measure trunk balance of the person under evaluation based on a movement of the person under evaluation produced by externally applying a force to the person under evaluation, and then an eyewear frame color providing a measurement result of good trunk balance may be determined.

To address the above issues, a twelfth aspect of the present invention provides a wearable thing color determination method for determining a color of a human wearable thing using the trunk balance measurement method according to any one of the first to fifth aspect of the present invention. The wearable thing color determination method includes: a color acquisition step of acquiring color information on the thing providing measurement results of good trunk balance in the measurement step; and a color determination step of determining a color of a wearable thing of the person under evaluation based on the color information acquired in the color acquisition step.

In the thirteenth aspect of the present invention, preferably, in the color determination step, a plurality of candidates for colors of a wearable thing may be selected based on the color information acquired in the color acquisition step, then the person under evaluation may be caused to have readily an unstable posture upon external application of a force to the person under evaluation, then the person under evaluation may be caused to wear a wearable thing with each color of the plurality of candidates in sequence, in order to measure trunk balance of the person under evaluation based on a movement of the person under evaluation produced by externally applying a force to the person under evaluation, and a color of the wearable thing providing a measurement result of good trunk balance may be determined.

### Advantageous Effects of Invention

According to the first aspect of the present invention, the trunk balance measurement method enables measurement of trunk balance of the person under evaluation based on the color of a thing. Therefore, the trunk balance measurement method can achieve a more suitable measurement of trunk balance of a person.

According to the second aspect of the present invention, in the trunk balance measurement method, by standing or sitting the person under evaluation on his/her foot on the measuring means, the posture of the person under evaluation of which trunk balance is to be measured may easily become unstable upon external application of a force to the person under evaluation.

According to the third, fourth aspects of the present invention, the measuring means is capable of standing the person under evaluation of which trunk balance is to be measured, such that the posture of the person under evaluation readily becomes more unstable upon external application of a force to the person under evaluation.

According to the fifth aspect of the present invention, the trunk balance measurement method enables more easy measurement of trunk balance because the things of different colors are colored paper sheets.

According to the sixth aspect of the present invention, the eyewear lens color determination method enables a determination of more suitable eyewear lens color for the person under evaluation by use of the results of trunk balance measurements according to the color of the thing.

According to the seventh aspect of the present invention, the eyewear lens color determination method enables a determination of more suitable eyewear lens color for the person under evaluation by use of the results of trunk balance measurements according to the eyewear lens color.

According to the eighth aspect of the present invention, the contact lens color determination method enables a determination of more suitable contact lens color for the person under evaluation by use of the results of trunk balance measurements according to the color of the thing.

According to the ninth aspect of the present invention, the contact lens color determination method enables a determination of more suitable contact lens color for the person under evaluation by use of the results of trunk balance measurements according to the contact lens color.

According to the tenth aspect of the present invention, the eyewear frame color determination method enables a determination of more suitable eyewear frame color for the person under evaluation by use of the results of trunk balance measurements according to the color of the thing.

According to the eleventh aspect of the present invention, the eyewear frame color determination method enables a determination of more suitable eyewear frame color for the person under evaluation by use of the results of trunk balance measurements according to the eyewear frame color.

According to the twelfth aspect of the present invention, the human wearable thing color determination method enables a determination of more suitable wearable thing color for the person under evaluation by use of the results of trunk balance measurements according to the color of the thing.

According to the thirteenth aspect of the present invention, the human wearable thing color determination method enables a determination of more suitable wearable thing color for the person under evaluation by use of the results of trunk balance measurements according to the color of the wearable thing.

### BRIEF DESCRIPTION OF DRAWINGS

FIG. 1 is a flow diagram illustrating an example of processing in a method for determining an eyewear lens color.

FIG. 2 is a flow diagram illustrating an example step of assessing trunk balance using colored paper.

FIG. 3 is a plan view illustrating an example shape of a plantar board.

FIG. 4 is a side view illustrating an example shape of the plantar board.

FIG. 5 is a diagram illustrating an example table for selecting candidates for lens color.

FIG. 6 is a flow diagram illustrating an example step of assessing trunk balance when eyewear with a candidate lens color is worn, and an example step of determining an eyewear lens color.

FIG. 7 is a flow diagram illustrating an example of processing in a method for determining a lens color of contact lenses.

FIG. 8 is a flow diagram illustrating an example step of assessing trunk balance when contact lenses with a candidate lens color are worn, and an example step of determining a lens color of the contact lenses.

FIG. 9 is a flow diagram illustrating an example of processing in a method for determining an eyewear frame color.

FIG. 10 is a diagram illustrating an example table for selecting candidates for frame color.

FIG. 11 is a flow diagram illustrating an example step of assessing trunk balance when eyewear with a candidate frame color is worn, and an example step of determining an eyewear frame color.

FIG. 12 is a block diagram illustrating an example configuration of a processing terminal.

FIG. 13 is a flowchart illustrating an example of the color determination processing for determining a lens color.

FIG. 14 is a diagram illustrating an example color selection operation performed on a color selection screen.

FIG. 15 is a diagram illustrating an example output screen.

FIG. 16 is a flow diagram illustrating an example of processing in the trunk balance measurement method.

### DESCRIPTION OF EMBODIMENTS

Embodiments according to the present invention will now be described with reference to the accompanying drawings.

### First Embodiment

In a first embodiment, an eyewear lens color determination method for determining an eyewear lens color will be provided.

### Configurations and Others

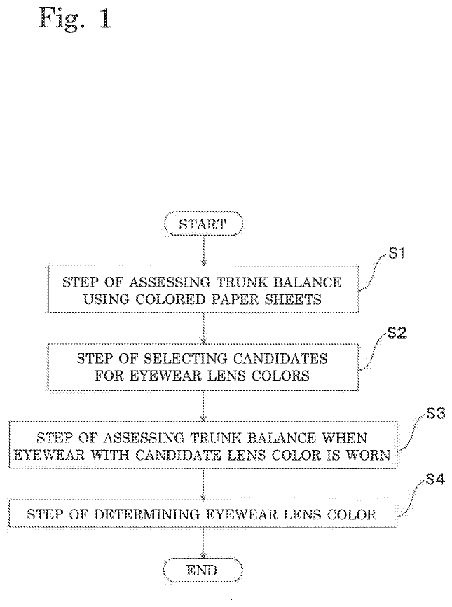

FIG. 1 is a flow diagram illustrating an example of processing in the method for determining an eyewear lens color.

As illustrated in FIG. 1, in the method for determining an eyewear lens color, a step of assessing trunk balance using colored paper sheets (step S1), a step of selecting candidates for eyewear lens colors (step S2), a step of assessing trunk balance when the eyewear with a candidate lens color is worn (step S3), and a step of determining an eyewear lens color (step S4) are performed in this order by operators in charge of the respective steps.

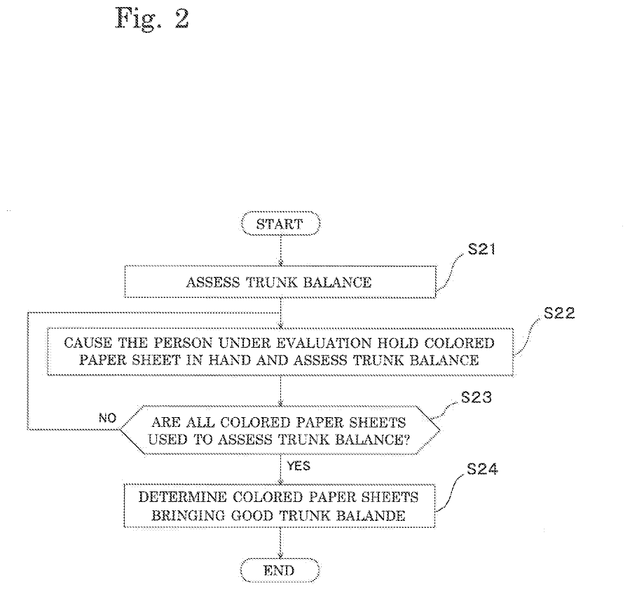

FIG. 2 is a flow diagram illustrating an example of the step of assessing trunk balance using colored paper sheets (step S1).

As illustrated in FIG. 2, in the steps, initially, as processing in step S21, the operator in charge assesses trunk balance of a person under evaluation without a colored paper sheet. Specifically, initially, the operator in charge lets the person under evaluation stand on one foot such that the person under evaluation places one of his/her feet (e.g., the left foot) on a plantar board, and keeps the other foot (e.g., the right foot) in the air rather than placing it on the plantar board. Hereinafter, the one foot will be referred to as a “foot on the plantar board”, and the other foot will be referred to as a “foot in the air”. Also, at this time, the person under evaluation may or may not wear his/her eyewear in current use during the assessment of trunk balance.

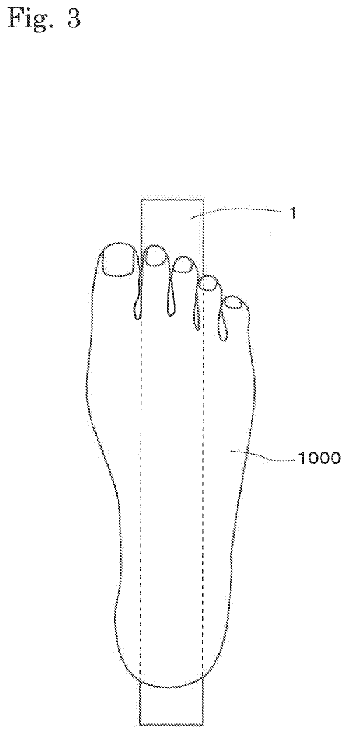

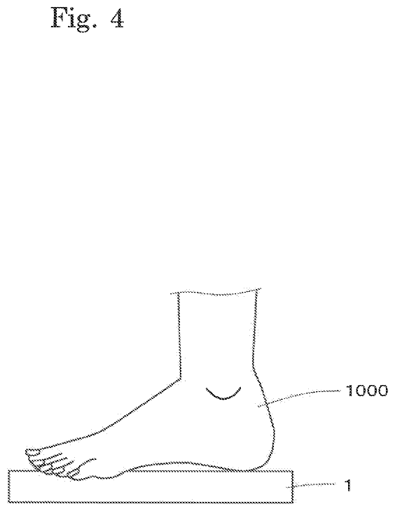

FIGS. 3 and 4 are views illustrating an example shape of the plantar board 1, in which FIG. 3 is a plan view and FIG. 4 is a side view.

As illustrated in FIGS. 3 and 4, the plantar board 1 is formed in an elongated rodlike shape of rectangular cross section, which has a shape extending from the heel to the toes of the foot 1000 and having a smaller width than that of the sole of the foot 1000 when the foot 1000 is placed thereon. For example, the plantar board 1 is made of wood, rubber or resins.

Then, the operator in charge pushes a shoulder (e.g., the right shoulder) on the same side of the foot in the air (e.g., the right foot) directly down. Then, trunk balance (e.g., deviation of the body axis) of the person under evaluation at this time is assessed. For example, the operator in charge has the person under evaluation assess his/her trunk balance for oneself.

In the processing in the subsequent step S22, the operator in charge has the person under evaluation hold the colored paper in his/her hand in this step, and then the operator in charge assesses trunk balance of the person under evaluation in a similar manner to step S21. Specifically, while the person under evaluation is holding a colored paper sheet of a selected color in his/her hand (e.g., left hand) on the same side of the foot on the plantar board, the operator in charge pushes a shoulder (e.g., right shoulder) on the same side of the foot in the air (e.g., right foot) directly down. Then, while the person under evaluation is looking at the colored paper sheet, the operator in charge has the person under evaluation assess his/her trunk balance (e.g., deviation of the body axis) at this time for oneself. At this time, the person under evaluation may or may not wear his/her eyewear in current use during the assessment of trunk balance.

In the processing at the subsequent step S23, the operator in charge checks whether or not all colored paper sheets are used to assess trunk balance of the person under evaluation. For example, the operator in charge prepares the colored paper sheets of six colors, i.e., red, blue, green, yellow, black, and white paper sheets. Thus, the operator in charge checks whether or not all the colored paper sheets are used to assess trunk balance of the person under evaluation. If all the colored paper sheets are not used to assess trunk balance of the person under evaluation, the operator in charge performs the processing from step S22 again so that the person under evaluation holds a colored paper sheet which has not yet been used to assess trunk balance of the person under evaluation in order to assess trunk balance of the person under evaluation. On the other hand, if all the colored paper sheets are not used to assess trunk balance of the person under evaluation, the operator in charge moves on to the processing at step $24.

In the processing at step S24, the operator in charge decides on the colored paper sheet brining good trunk balance. Specifically, the operator in charge has the person under evaluation select two colored paper sheets brining good trunk balance from among the all the colored paper sheets used to assess trunk balance. For example, the person under evaluation selects the colored paper sheets of two colors providing better trunk balance in comparison with the trunk balance when he/she holds no colored paper sheet (trunk balance in step S21), and/or comparison with the trunk balance when he/she holds another colored paper sheet. The operator in charge determines the colored paper sheets thus selected by the person under examiner as colored paper sheets brining good trunk balance.

As described above, in the step of assessing trunk balance using the colored paper sheets, the operator in charge assesses trunk balance of the person under evaluation who stands on one foot on the plantar board. Then, the operator in charge has the person under evaluation hold a colored paper sheet in his/her hand while the person under evaluation is standing on one foot on the plantar board, in order to assess trunk balance of the person under evaluation. After trunk balance of the person under evaluation has been assessed using all the colored paper sheets, the operator in charge determines the colored paper sheet(s) brining good trunk balance.

In the subsequent step of selecting candidates for eyewear lens colors (step S2), the operator in charge selects candidates for eyewear lens colors (candidate lens colors) based on the colored paper sheets that have been selected (determined) in the step of assessing trunk balance using the colored paper sheets (step S1). Specifically, the operator in charge consults a table for selecting candidates for lens color to determine candidates for eyewear lens color.

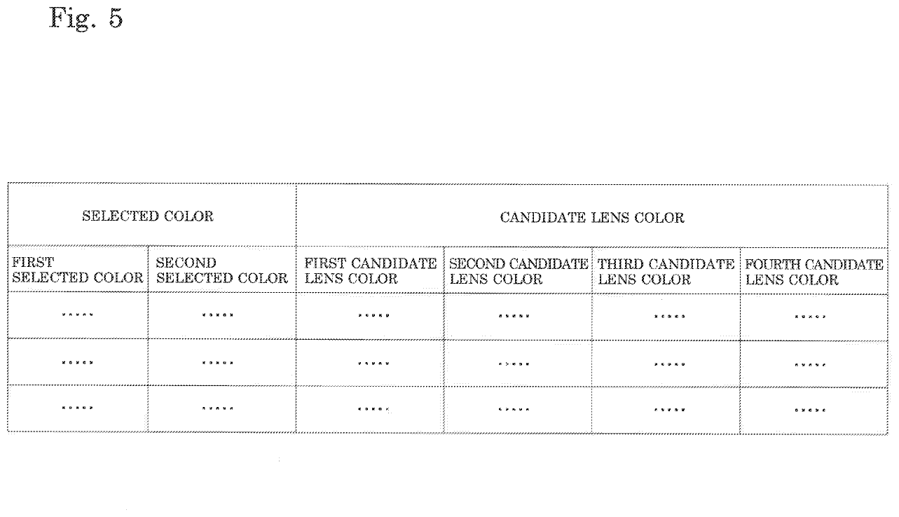

FIG. 5 is a diagram illustrating an example table for selecting candidates for lens color.

As illustrated in FIG. 5, in the lens color candidate selection table, a first selected color and a second selected color are associated with first to fourth candidate lens colors, in which a first candidate lens color is a color corresponding to a first selected color. For example, the first candidate lens color is the same color as or a similar color to the first selected color, or alternatively, is a preset color. Also, the second candidate lens color is a color corresponding to the second selected color. For example, the second candidate lens color is the same color as or a similar color to the second selected color, or alternatively, is a preset color. Also the third and fourth candidate lens colors are two colors selected from among combinations of the first selected color and the second selected color. And, the first to fourth candidate lens colors are reproducible colors as actual eyewear lens color. For example, each of the first selected color and the second selected color is any of red, blue, green, yellow, black and white colors. In such cases, in the lens color candidate selection table, combinations of the first selected color which is any of red, blue, green, yellow, black and white colors and the second selected color which is any of red, blue, green, yellow, black and white colors are associated with the first to fourth candidate lens colors. The first to fourth candidate lens colors are configured by an empirical value, a calculated value, a theoretical value and/or the like, or alternatively are configured statistically.

The operator in charge consults the lens color candidate selection table using, as a first selected color or a second selected color, the colors of the two colored paper sheets selected in the step of assessing trunk balance using colored paper sheets (step S1 or step S24), and then the operator in charge selects first to fourth candidate lens colors associated with the first selected color and the second selected color.

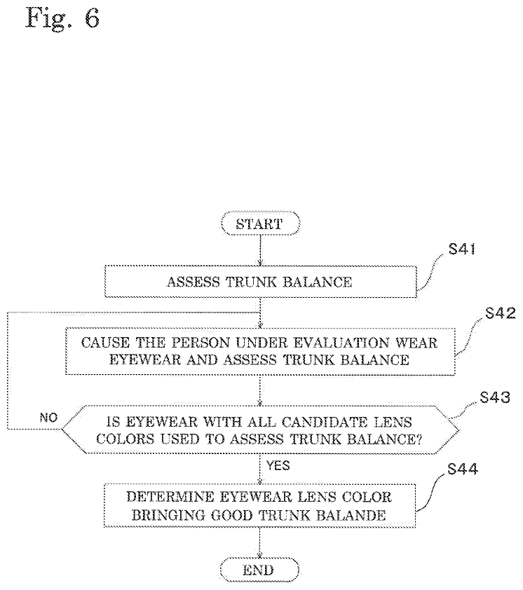

FIG. 6 is a flow diagram illustrating an example step of assessing trunk balance when eyewear with a candidate lens color is worn (step S3), and an example step of determining an eyewear lens color (step S4).

As illustrated in FIG. 6, in the steps, initially, as processing in step S41, similar to step S21, the operator in charge has the person under evaluation stand on one foot on the plantar board, then pushes directly down a shoulder (e.g., the right shoulder) of the person under evaluation on the same side of the foot in the air (e.g., the right foot), and the operator in charge then assesses trunk balance of the person under evaluation at this time. At this time, the person under evaluation may or may not wear his/her eyewear in current use during the assessment of trunk balance.

As processing at the subsequent step S42, the operator in charge has the person under evaluation, who stands on one foot on the plantar board, wear eyewear with a candidate lens color selected in the step of selecting candidates for eyewear lens color (step S2), and then the operator in charge assesses trunk balance of the person under evaluation. For example, the operator in charge pushes directly down a shoulder (e.g., the right shoulder) of the person under evaluation on the same side of the foot in the air (e.g., the right foot), and then has the person under evaluation assess his/her trunk balance for oneself at this time.

In the processing at the subsequent step S43, the operator in charge checks whether or not all candidate lens colors for eyewear are used to assess trunk balance of the person under evaluation. For example, the operator in charge checks whether or not all the candidate lens colors (the first to fourth candidate lens colors) selected as candidates in the step of selecting candidates for eyewear lens color are used to assess trunk balance of the person under evaluation. If all the candidate lens colors are not used to assess trunk balance of the person under evaluation, the operator in charge performs the processing from step S42 again and has the person under evaluation wear eyewear with candidate lens colors which have not yet been used for the assessment of trunk balance of the person under evaluation, in order to assess trunk balance of the person under evaluation. On the other hand, if all the candidate lens colors for eyewear are used to assess trunk balance of the person under evaluation, the operator in charge moves on to the processing at step S44.

In the processing at step S44, the operator in charge decides on a lens color brining good trunk balance. Specifically, the operator in charge has the person under evaluation select one or two lens colors bringing good trunk balance from among all the candidate lens colors used to assess trunk balance. For example, the person under evaluation selects the lens color(s) bringing better trunk balance in comparison with trunk balance when he/she wears no eyewear with a candidate lens color (trunk balance in step S41), and/or in comparison with trunk balance when he/she wears eyewear with another candidate lens color. Then, the operator in charge determines the candidate lens color thus selected by the person under examiner as a lens color bringing good trunk balance.

As described above, in the step of assessing trunk balance when the eyewear is worn and the step of determining an eyewear lens color, the operator in charge assesses trunk balance of the person under evaluation who stands on one foot on the plantar board. Then, the operator in charge has the person under evaluation wear eyewear with the candidate lens color while the person under evaluation is standing on one foot on the plantar board, in order to assess trunk balance of the person under evaluation. Then, after trunk balance of the person under evaluation has been assessed using all the candidate lens colors, the operator in charge determines a lens color of the candidate lens colors that brings good trunk balance.

### Working and Others

An example of working and the like in the method for determining an eyewear lens color will now be described.

In the step of assessing trunk balance using the colored paper sheets, an operator in charge assesses trunk balance of a person under evaluation standing on one foot on the plantar board. The operator in charge then has the person under evaluation who is standing on one foot on the plantar board hold a colored paper sheet in his/her hand, and thus the operator in charge assesses trunk balance of the person under evaluation. Then, the operator in charge assesses trunk balance of the person under evaluation using all the colored paper sheets, and then determines colored paper sheets of them bringing good trunk balance.

Subsequently, in the step of selecting candidates for eyewear lens color, the operator in charge selects candidates for eyewear lens color based on the colors of the precedingly determined colored paper sheets.

Subsequently, in the step of assessing trunk balance when eyewear with a candidate lens color is worn, and the step of determining an eyewear lens color, the operator in charge assesses trunk balance of the person under evaluation standing on one foot on the plantar board. Then, the operator in charge has the person under evaluation wear the eyewear while the person under evaluation is standing on one foot on the plantar board, and thus the operator in charge assesses the trunk balance of the person under evaluation. And, after using all the candidate lens colors to assess trunk balance of the person under evaluation, the operator in charge determines a lens color of the candidate lens colors that brings good trunk balance. Then, the operator in charge makes eyewear with the lens color thus determined, specifically for the person under evaluation.

Here, a relationship between the first and second selected colors and the first to fourth candidate lens colors associated with them in the lens color candidate selection table is established in which, when a person under evaluation selecting the first selected color and the second selected color wears eyewear using the first to fourth candidate lens colors associated with the selected colors, this increases the possibility that the eyewear lens colors match the body of the person under evaluation. Therefore, the eyewear thus made will match the body of the person under evaluation.

### Advantageous Effects in First Embodiment

1. In the method for determining an eyewear lens color, trunk balance of a person under evaluation is able to be measured based on color of a colored paper sheet. This enables more optimum measurement of person's trunk balance in the method for determining an eyewear lens color.

2. In the method for determining an eyewear lens color, standing a person under evaluation on foot on the plantar board enables making the posture of the person under evaluation readily unstable when a force is externally applied to the person under evaluation of which the trunk balance is to be measured.

3. The plantar board has a shape for a person under evaluation to place his/her foot thereon, the shape extending from the heel to the toes of the foot and having a smaller width than that of the sole of the foot. By virtue of this, the person under evaluation undergoing a measurement of trunk balance may stand such that his/her posture readily becomes more unstable upon external application of a force thereto.

4. In the method for determining an eyewear lens color, using the colored paper sheets may further facilitate the measurement of trunk balance.

5. In the method for determining an eyewear lens color, using the results of trunk balance measurements according to colors of the colored paper sheets enables the determination of more suitable eyewear lens color for a person under evaluation.

6. In the method for determining an eyewear lens color, using the results of trunk balance measurements according to lens colors enables the determination of more suitable eyewear lens color for a person under evaluation.

7. In the method for determining an eyewear lens color, it is possible to determine more suitable eyewear lens color for a person under evaluation based on information on two different colors.

### Second Embodiment

In a second embodiment, a contact-lens lens color determination method for determining a lens color of a contact lens will be provided. The eyewear lens color is determined in the first embodiment, and a lens color of a contact lens is determined in the second embodiment.

### Configuration and Others

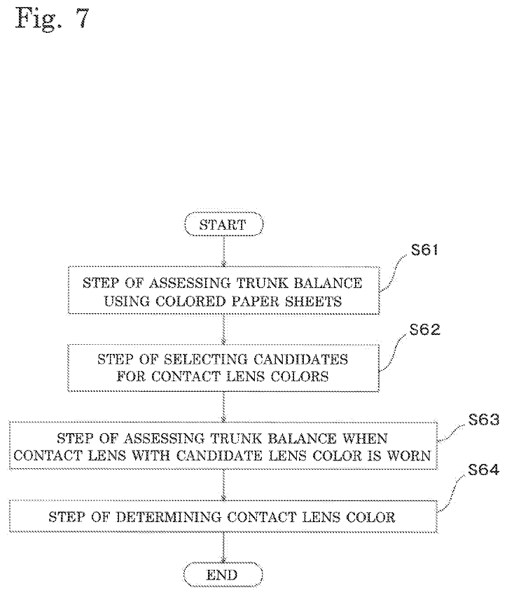

FIG. 7 is a flow diagram illustrating an example of processing in a method for determining a lens color of a contact lens.

As illustrated in FIG. 7, in the method for determining a lens color of a contact lens, as in the case of the method for determining an eyewear lens color, a step of assessing trunk balance using colored paper sheets (step S61), a step of selecting candidates for contact-lens lens color (step S62), a step of assessing trunk balance when a contact lens with a candidate lens color is worn (step S63), and a step of determining a contact-lens lens color (step S64) are performed in this order by operators in charge of the respective steps.

The details of the step of assessing trunk balance using colored paper sheets (step S61) are the same as the step of assessing trunk balance using colored paper sheets (step S1) in the first embodiment. In the subsequent step of selecting candidates for contact-lens lens color (step S62), the operator in charge selects a lens color for a contact lens based on the colored paper sheets selected (determined) in the step of assessing trunk balance using the colored paper sheets. Specifically, the lens color candidate selection table is consulted to select candidates for contact-lens lens color. For example, the lens color candidate selection table may be the lens color candidate selection table used in the determination of the eyewear lens color in the first embodiment, or otherwise may be a lens color candidate selection table separately prepared for selection of contact-lens lens color.

The operator in charge consults the lens color candidate selection table using colors of the two colored paper sheets selected in the step of assessing trunk balance using colored paper as a first selected color and a second selected color. And then, the operator in charge selects, as candidates, first to fourth lens colors associated with the first selected color and the second selected color. Then, the operator in charge moves on to processing in a step of assessing trunk balance when the contact lens is worn (step S63), and a step of determining a contact-lens lens color (step S64).

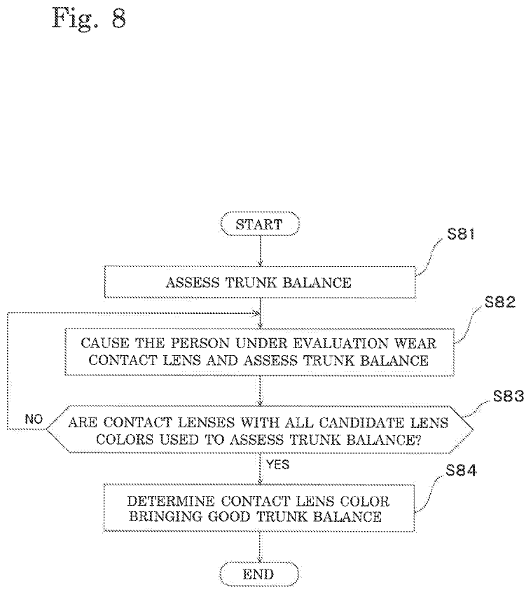

FIG. 8 is a flow diagram illustrating an example step of assessing trunk balance when a contact lens is worn (step S63), and an example step of determining a contact-lens lens color (step S64).

Specifically, as illustrated in FIG. 8, in the steps, initially, as processing at step S81, similar to step S21, the operator in charge has the person under evaluation stand on one foot on the plantar board, then pushes directly down a shoulder (e.g., the right shoulder) of the person under evaluation on the same side of the foot in the air (e.g., the right foot), and the operator in charge then assesses trunk balance of the person under evaluation at this time.

As processing at the subsequent step S82, after the person under evaluation standing on one foot on the plantar board wears contact lenses of a candidate lens color selected in the step of selecting candidates for contact-lens lens color, the operator in charge assesses trunk balance of the person under evaluation. For example, the operator in charge pushes directly down a shoulder (e.g., the right shoulder) of the person under evaluation on the same side of the foot in the air (e.g., the right foot), and the operator in charge has the person under evaluation assess his/her trunk balance for oneself at this time.

In the processing at the subsequent step S83, the operator in charge checks whether or not all the candidate lens colors of the contact lens are used to assess trunk balance of the person under evaluation. For example, the operator in charge checks whether or not all the candidate lens colors (the first to fourth candidate lens colors) selected in the step of selecting candidates for contact-lens lens colors are used to assess trunk balance of the person under evaluation. If all the candidate lens colors are not used to assess trunk balance of the person under evaluation, the operator in charge performs the processing from step S82 again so that the person under evaluation wears contact lenses with candidate lens colors which have not yet been used to assess the trunk balance of the person under evaluation in order to assess the trunk balance of the person under evaluation. On the other hand, if all the candidate lens colors of contact lenses are used to assess trunk balance of the person under evaluation, the operator in charge moves on to the processing at step S84.

In the processing at step S84, the operator in charge determines a lens color bringing good trunk balance. Specifically, the operator in charge lets the person under evaluation select one or two candidate lens colors bringing good trunk balance from all the candidate lens colors used to assess trunk balance. For example, the person under evaluation selects lens colors bringing good trunk balance in comparison with trunk balance when no contact lens with the candidate lens color is worn (trunk balance in step S81), and/or with trunk balance when contact lenses with another candidate lens color are worn. Then, the operator in charge determines the candidate lens color thus selected by the person under examiner as a lens color bringing good trunk balance.

As described above, in the step of assessing the trunk balance when the contact lenses are worn and the step of determining the contact-lens lens color, the operator in charge assesses trunk balance of the person under evaluation who stands on one foot on the plantar board. Then, the operator in charge lets the person under evaluation wear contact lenses with the candidate lens color while the person under evaluation is standing on one foot on the plantar board, in order to assess trunk balance of the person under evaluation. After the trunk balance of the person under evaluation has been assessed using all the candidate lens colors, the operator in charge determines a lens color of the candidate lens colors that brings good trunk balance.

### Working and Others

An example of working and the like in the method for determining a lens color of a contact lens will now be described.

In the step of assessing trunk balance using the colored paper sheets, the operator in charge assesses trunk balance of a person under evaluation standing on one foot on the plantar board. The operator in charge then lets the person under evaluation stand on one foot on the plantar board and hold a colored paper sheet, and thus the operator in charge assesses trunk balance of the person under evaluation. Then, after using all the colored paper sheets to assess trunk balance of the person under evaluation, the operator in charge determines colored paper sheets of them bringing good trunk balance.

Subsequently, in the step of selecting candidates for contact-lens lens color, the operator in charge selects candidates for contact lens color based on the colors of the precedingly determined colored paper sheets.

Subsequently, in the step of assessing trunk balance when a contact lens(es) with a candidate lens color is worn, and the step of determining a lens color of the contact lens, the operator in charge assesses trunk balance of the person under evaluation standing on one foot on the plantar board. Then, the operator in charge lets the person under evaluation wear the contact lens(es) while standing on one foot on the plantar board, and thus the operator in charge assesses trunk balance of the person under evaluation. And, after using all the candidate lens colors to assess trunk balance of the person under evaluation, the operator in charge determines a lens color of the candidate lens colors that brings good trunk balance. Then, the operator in charge makes contact lenses with the lens color thus determined, specifically for the person under evaluation.

Here, a relationship between the first and second selected colors in the lens color candidate selection table and the first to fourth candidate lens colors associated with them is established in which, when a person under evaluation selecting the first selected color and the second selected color wears contact lenses with the first to fourth candidate lens colors associated with the selected colors in question, this increases the possibility that the lens color of the contact lenses in question matches the body of the person under evaluation in question. Therefore, the contact lenses thus made will match the body of the person under evaluation.

### Advantageous Effects in Second Embodiment

1. In the method for determining a lens color of a contact lens, similar advantageous effects to those in the method for determining an eyewear lens color are produced.

2. In the method for determining a lens color of a contact lens, using the results of trunk balance measurements according to colors of the colored paper sheets enables the determination of more suitable contact-lens color for a person under evaluation.

3. In the method for determining a lens color of a contact lens, it is possible to determine more suitable contact lens color for a person under evaluation based on information on two different colors.

### Third Embodiment

In a third embodiment, an eyewear frame color determination method for determining an eyewear frame color will be provided. The lens color is determined in the first and second embodiments, and a frame color is determined in the third embodiment.

### Configuration and Others

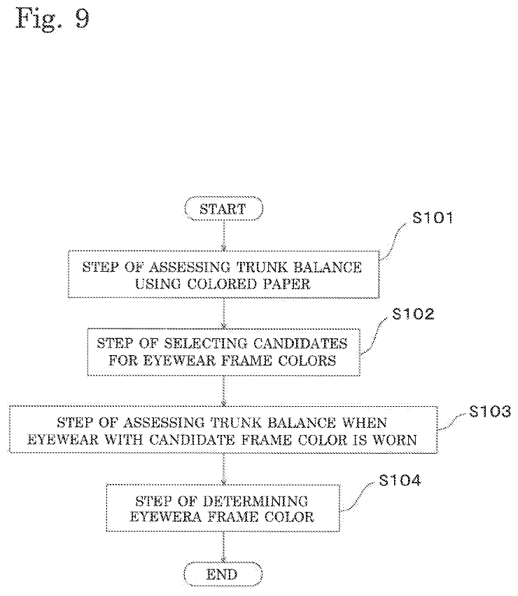

FIG. 9 is a flow diagram illustrating an example of processing in a method for determining an eyewear frame color.

As illustrated in FIG. 9, in the method for determining an eyewear frame color, a step of assessing trunk balance using colored paper sheets (step S101), a step of selecting candidates for eyewear frame color (step S102), a step of assessing trunk balance when eyewear with a candidate frame color is worn (step S103), and a step of determining an eyewear frame color (step S104) are performed in this order by operators in charge of the respective steps.

The details of the step of assessing trunk balance using colored paper sheets (step S101) are the same as the step of assessing trunk balance using colored paper sheets (step S1) in the first embodiment. In the subsequent step of selecting candidates for eyewear frame color (step S102), the operator in charge selects an eyewear frame color based on the colored paper sheets selected (determined) in the step of assessing trunk balance using the colored paper sheets. Specifically, the frame color candidate selection table is referred to select candidates for eyewear frame color.

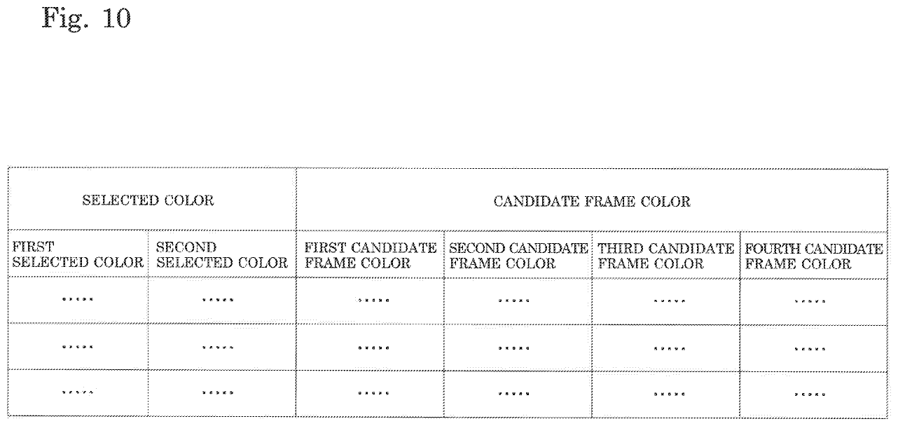

FIG. 10 is a diagram illustrating an example table for selecting candidates for frame color.

As illustrated in FIG. 10, in the frame color candidate selection table, a first selected color and a second selected color are associated with first to fourth candidate frame colors, in which a first candidate frame color is a color corresponding to a first selected color. For example, the first candidate frame color is the same color as or a similar color to the first selected color, or alternatively, is a preset color. Also, the second candidate frame color is a color corresponding to the second selected color. For example, the second candidate frame color is the same color as or a similar color to the second selected color, or alternatively, is a preset color. Also, the third and fourth candidate frame colors are two colors selected from combinations of the first selected color and the second selected color. And, the first to fourth candidate frame colors are reproducible colors as actual eyewear frame color. For example, each of the first selected color and the second selected color is any of red, blue, green, yellow, black and white colors. In such cases, in the frame color candidate selection table, combinations of the first selected color which is any of red, blue, green, yellow, black and white colors and the second selected color which is any of red, blue, green, yellow, black and white colors are associated with the first to fourth candidate frame colors. The first to fourth candidate frame colors are configured by an empirical value, a calculated value, a theoretical value and/or the like, or alternatively are configured statistically.

The operator in charge refers the frame color candidate selection table using, as a first selected color or a second selected color, the colors of the two colored paper sheets selected in the step of assessing trunk balance using colored paper sheets, and then the operator in charge selects, as candidates, first to fourth candidate frame colors associated with the first selected color and the second selected color. Then, the operator in charge moves on to processing at the step of assessing trunk balance when eyewear is worn (step S103) and at the step of determining an eyewear frame color (step S104).

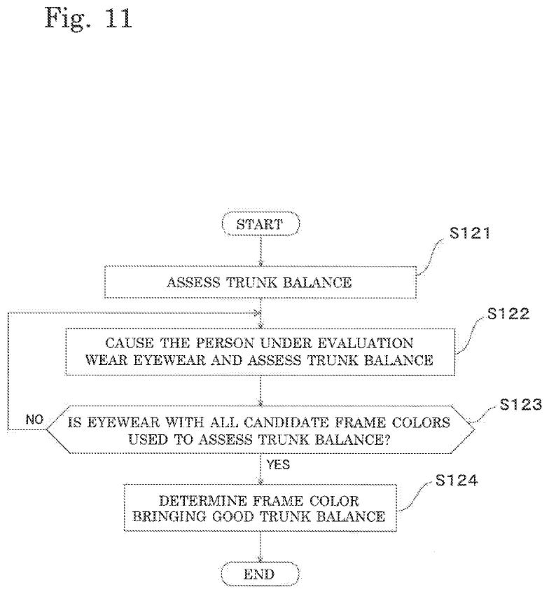

FIG. 11 is a flow diagram illustrating an example step of assessing trunk balance when eyewear is worn (step S103), and an example step of determining an eyewear frame color (step S104).

As illustrated in FIG. 11, in the steps, initially, as processing in step S121, similar to step S21, the operator in charge lets the person under evaluation stand on one foot on the plantar board, then pushes directly down a shoulder (e.g., the right shoulder) of the person under evaluation on the same side of the foot in the air (e.g., the right foot), and the operator in charge then assesses trunk balance of the person under evaluation at this time.

As processing at the subsequent step S122, after the person under evaluation standing on one foot on the plantar board wears eyewear with a candidate frame color selected in the step of selecting candidates for eyewear frame color, the operator in charge assesses trunk balance of the person under evaluation. For example, the operator in charge pushes directly down a shoulder (e.g., the right shoulder) of the person under evaluation on the same side of the foot in the air (e.g., the right foot), and the operator in charge lets the person under evaluation assess his/her trunk balance for oneself at this time. Here, the eyewear may or may not fitted with lenses. Also, if the eyewear is fitted with a lens, the lens may be a lens of eyewear of the person under evaluation own, a temporary lens prepared by the operator in charge or lens finally determined in the first embodiment.

In the processing at the subsequent step S123, the operator in charge checks whether or not all candidate frame colors of the eyewear are used to assess trunk balance of the person under evaluation. For example, the operator in charge checks whether or not all the candidate frame colors (the first to fourth candidate frame colors) selected in the step of selecting candidates for eyewear frame color are used to assess trunk balance of the person under evaluation. If all the candidate frame colors are not used to assess trunk balance of the person under evaluation, the operator in charge performs the processing from step S122 again, in which the operator in charge lets the person under evaluation wear eyewear with a candidate frame color which has not yet been used to assess trunk balance of the person under evaluation, for assessment of trunk balance of the person under evaluation. On the other hand, if all the candidate frame colors of eyewear are used to assess trunk balance of the person under evaluation, the operator in charge moves on to the processing at step S124.

In the processing at step S124, the operator in charge decides on a frame color bringing good trunk balance. Specifically, the operator in charge selects one or two candidate frame colors bringing good trunk balance from all the candidate frame colors used to assess trunk balance. For example, the person under evaluation selects frame colors bringing good trunk balance in comparison with trunk balance when no eyewear with the candidate frame color is worn (trunk balance in step S121), and/or in comparison with trunk balance when eyewear with another candidate frame color is worn. Then, the operator in charge determines the candidate frame colors thus selected by the person under examiner as a frame color bringing good trunk balance.

As described above, in the step of assessing the trunk balance when the eyewear is worn and the step of determining the eyewear frame color, the operator in charge assesses trunk balance of the person under evaluation who stands on one foot on the plantar board. Then, the operator in charge lets the person under evaluation stand on one foot on the plantar board and wear eyewear with the candidate lens color, and the operator in charge assesses trunk balance of the person under evaluation. Then, after trunk balance of the person under evaluation has been assessed using all the candidate frame colors, the operator in charge determines a frame color of the candidate frame colors that brings good trunk balance.

### Working and Others

An example of working and the like in the method for determining an eyewear frame color will now be described.

In the step of assessing trunk balance using the colored paper sheets, the operator in charge assesses trunk balance of a person under evaluation standing on one foot on the plantar board. The operator in charge then lets the person under evaluation stand on one foot on the plantar board and hold a colored paper sheet, and thus the operator in charge assesses trunk balance of the person under evaluation. After using all the colored paper sheets to assess trunk balance of the person under evaluation, the operator in charge determines colored paper sheets of them bringing good trunk balance.

Subsequently, in the step of selecting candidates for eyewear frame color, the operator in charge selects candidates for contact lens color based on the colors of the precedingly determined colored paper sheets.

Subsequently, in the step of assessing trunk balance when eyewear with a candidate frame color is worn, and the step of determining an eyewear frame color, the operator in charge assesses trunk balance of the person under evaluation standing on one foot on the plantar board. Then, the operator in charge lets the person under evaluation wear the eyewear while standing on one foot on the plantar board, and thus the operator in charge assesses trunk balance of the person under evaluation. And, after using all the candidate frame colors to assess trunk balance of the person under evaluation, the operator in charge determines a frame color of the candidate lens colors that brings good trunk balance. Then, the operator in charge makes eyewear with the frame color thus determined, specifically for the person under evaluation.

Here, a relationship between the first and second selected colors in the frame color candidate selection table and the first to fourth candidate frame colors associated with them is established in which, when a person under evaluation selecting the first selected color and the second selected color wears eyewear with the first to fourth candidate frame colors associated with the selected colors in question, this increases the possibility that the frame color of the eyewear in question matches the body of the person under evaluation in question. Therefore, the eyewear thus made will match the body of the person under evaluation.

### Advantageous Effects in Third Embodiment

1. In the method for determining an eyewear frame color, similar advantageous effects to those in the method for determining an eyewear lens color are produced.

2. In the method for determining an eyewear frame color, using the results of trunk balance measurements according to colors of the colored paper sheets enables the determination of more suitable eyewear frame color for a person under evaluation.

3. In the method for determining an eyewear frame color, it is possible to determine more suitable eyewear frame color for a person under evaluation based on information on two different colors.

### Fourth Embodiment

In a fourth embodiment, a processing terminal used in the method for determining an eyewear lens color will be provided.

### Configuration and Others

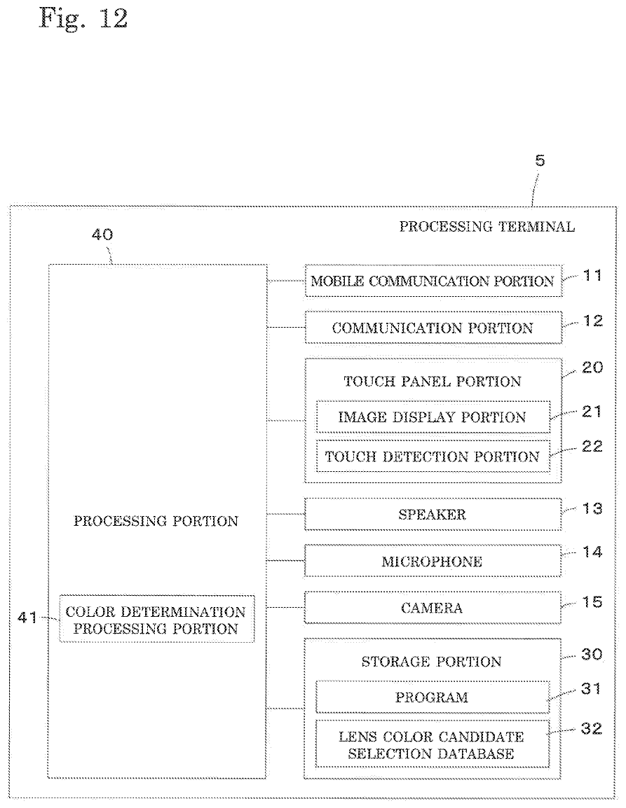

FIG. 12 is a block diagram illustrating an example configuration of a processing terminal 5. For example, the processing terminal 5 is a mobile phone.

As illustrated in FIG. 12, the processing terminal 5 has a mobile communication portion 11, a communication portion 12, a touch panel portion 20, a speaker 13, a microphone 14, a camera 15, a storage portion 30 and a processing portion 40. Here, the mobile communication portion 11 functions as performing telephone communication. The communication portion 12 performs wired or wireless communication with devices such as a server and/or the like over an Internet connection and/or the like. The touch panel portion 20 has an image display portion 21 and a touch detection portion 22, the image display portion 21 serving an image display function in the touch panel portion 20, the touch detection portion 22 serving an information input function. The storage portion 30 is, for example, ROM, RAM and/or the like. The processing portion 40 executes a variety of processing in the processing terminal 5. The processing portion 40 includes, for example, a microcomputer and the surrounding circuitry, and is essentially made up of, for example, CPU, ROM, RAM and/or the like. ROM has one or two or more programs stored therein. CPU executes a variety of processing in accordance with one or two or more programs stored in ROM. And, as required, the processing portion 40 executes a variety of processing in accordance with a variety of data and/or various programs stored in the storage portion 30. For example, a program 31 stored in the storage portion 30 is an application program. In the example, further, in the storage portion 30, the lens color candidate selection table illustrated in FIG. 5 is stored as a lens color candidate selection database 32. The processing portion 40 has a color determination processing portion 41 consulting the lens color candidate selection database 32 to execute color determination processing.

The color determination processing portion 41 consults the lens color candidate selection database 32 to execute color determination processing.

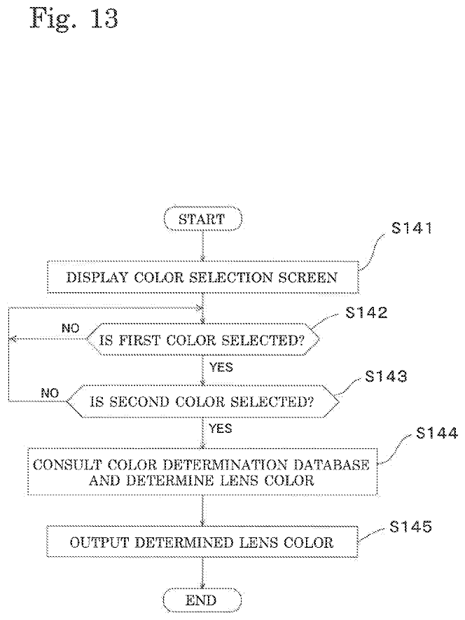

FIG. 13 is a flowchart illustrating an example of the color determination processing for determining a lens color.

As illustrated in FIG. 13, initially, as processing in step S141, the color determination processing portion 41 displays a color selection screen on the image display portion 21.

In the processing at the subsequent step S142, the color determination processing portion 41 determines whether or not a first color is selected on the color selection screen through the operation of a user (operator un charge). If determining that the first color is selected, the color determination processing portion 41 moves on to the processing at step S143.

In the processing at step S143, the color determination processing portion 41 determines whether or not a second color is selected on the color selection screen through the operation of the user. If determining that the second color is selected, the color determination processing portion 41 moves on to the processing at step S144. On the other hand, if determining that the second color is not selected, the color determination processing portion 41 returns to the processing at step S142. Through the processing in step S142 and step S143, the color determination processing portion 41 causes the user to select the first color and the second color on the color selection screen.

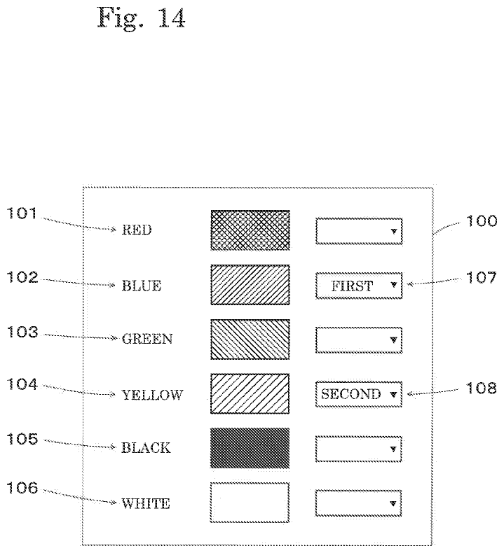

FIG. 14 is a diagram illustrating an example color selection operation performed on a color selection screen 100.

As illustrated in FIG. 14, red, blue, green, yellow, black and white color indications 101, 102, 103, 104, 105 and 106 are displayed on the color selection screen 100, and values representing priority order are displayed at the side of each of the color indications 101 to 106 in a selectable fashion. For example, values 107, 108 representing “first”, “second” can be displayed in a pulldown menu. In the example in FIG. 14, the blue color indication 102 is assigned the “first” value 107, so that it is selected as a first color, and the yellow color indication 104 is assigned the “second” value 108, so that it is selected as a second color. As a result, the color determination processing portion 41 acquires blue information as the first color and yellow information as the second color.

In the processing at step S144, the color determination processing portion 41 consults the lens color candidate selection database 32 using the first color and the second color selected through the processing in step S142 and step S143 respectively as a first selected color and a second selected color, and then the color determination processing portion 41 determines, as eyewear lens colors (candidates for lens color), four lens colors which are first to fourth candidate lens colors corresponding to the first selected color and the second selected color. For example, if colors as illustrated in FIG. 14 are selected, the color determination processing portion 41 consults the lens color candidate selection database 32 to determine the first to fourth candidate lens colors associated with combinations of blue and yellow colors as eyewear lens colors.

In the processing at the subsequent step S145, the color determination processing portion 41 outputs the four lens colors (candidate lens colors) determined through the processing in the step S144 to the image display portion 21.

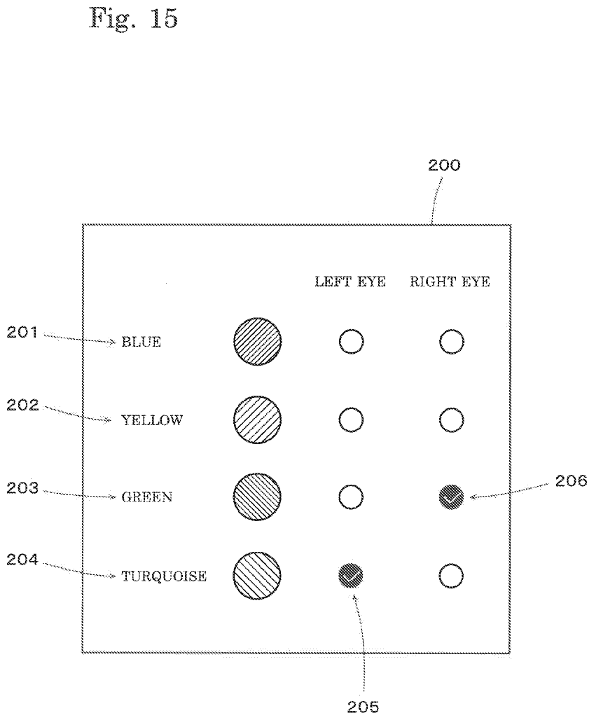

FIG. 15 is a diagram illustrating an example output screen 200.

As illustrated in FIG. 15, on the output screen 200, indications 201, 202, 203, 204 of the determined four lens colors are displayed. In the example illustrated in FIG. 15, on the output screen 200, the indications 201, 202, 203, 204 of four blue, yellow, green and turquoise colors are displayed as the determined lens colors (candidate lens colors). For example, on the output screen 200, the determined four lens colors are arranged and displayed in the order from top to bottom of a first candidate lens color, a second candidate lens color, a third candidate lens color, and a fourth candidate lens color.

And, as illustrated in FIG. 15, the output screen 200 is configured to show checkboxes arranged parallel to the determined four lens colors such that the user can operate the processing terminal 5 to check the checkbox for selection of a lens color for each of the left and right eyes. In the example, the checkbox 205 at the side of the turquoise indication 204 is checked, and the checkbox 206 at the side of the green indication 203 is checked. This display means when the user selects turquoise as a lens color for the left eye and green as a lens color for the right eye.

### Working and Others

An example of operation of the processing terminal 5 and an example of the working and the like will now be described.

In the processing terminal 5, when the user selects the first color and the second color on the color selection screen (see FIG. 14) displayed on the image display portion 21, the color determination processing portion 41 consults the lens color candidate selection database 32 so that the first to fourth candidate lens colors associated with the first color (first selected color) and second color (second selected color) which have been thus selected are displayed on the output screen (see FIG. 15). And, the output screen is configured to allow lens colors for the left and right eyes to be selected in an area at the side of the determined four lens colors.

Thus, on the color selection screen displayed on the image display portion 21 of the processing terminal 5, the user (operator in charge) may select the first color and the second color which are selected in the step of assessing trunk balance using the colored paper sheets. In the processing terminal 5, upon selection of the first color and the second color on the color selection screen, the lens color candidate selection database 32 is consulted to display the first to fourth candidate lens colors associated with the selected first and second colors on the output screen. Then, the user performs the step of assessing trunk balance when eyewear is worn, based on the first to fourth candidate lens colors displayed on the output screen. Thus, in the step of determining an eyewear lens color, the lens colors for the left and right eyes may be selected on the output screen. This enables the user to request a maker to make eyewear with designated lens color finally selected on the output screen.

It is noted that, in the description of the above-described embodiments, the planter board essentially forms, for example, measuring means. Also, the colored paper sheets essentially form, for example, things of different colors. Also, the processing in step S21, step S22 and/or the like for assessment of trunk balance essentially forms, for example, an external force input step and a measurement step. Also, the processing in the step of assessing trunk balance using colored paper sheets essentially forms, for example, a color acquisition step. Also, the step of determining an eyewear lens color essentially forms, for example, a color determination step.

### Modifications and Others of the Embodiments

As a modification of the above-described embodiments, the shape of the plantar board may be formed in a shape different from the above-mentioned shape, as long as the person under evaluation of which trunk balance is to be measured can stand such that his/her posture easily becomes more unstable when a force is externally applied thereto.

Further, in a modification of the above-described embodiments, in the method for determining an eyewear lens color, the method for determining a lens color for a contact lens, and the method for determining an eyewear frame color, instead of the plantar board, a tool, a device or the like may be used to allow the person under evaluation of which trunk balance is to be measured to stand such that his/her posture easily becomes more unstable when a force is externally applied thereto. For example, the tool, device or the like is a device horizontally vibrating. Alternatively, the tool, device or the like is a cushion member.

Further, in a modification of the above-described embodiments, in the method for determining an eyewear lens color, the method for determining a lens color for a contact lens, and the method for determining an eyewear frame color, trunk balance may be measured with the person under evaluation standing on both feet or being seated. Further, in a modification of the above-described embodiments, the measurement of trunk balance may be made by applying an external force to a part of the person under evaluation other than a shoulder.

Further, in a modification of the above-described embodiments, rather than the colored paper sheets, colored files, vessels containing colored liquid, or the like may be used as things having different colors.

Further, in a modification of the above-described embodiments, in the method for determining an eyewear lens color, the method for determining a lens color for a contact lens, and the method for determining an eyewear frame color, trunk balance of the person under evaluation may be objectively evaluated and measured in terms of physical movements of the person under evaluation and/or the like by the operator in charge and/or a device, rather than the person under evaluation oneself.

Further, in a modification of the above-described embodiments, the operator in charge may simply measure trunk balance of the person under evaluation without the purpose of determining an eyewear lens color, a lens color for a contact lens or an eyewear frame color. Also, the operator in charge may determine a color of a thing, rather than an eyewear lens, a contact lens or an eyewear frame, for the person under evaluation based on trunk balance of the person under evaluation.

Further, in a modification of the above-described embodiments, in the step of selecting candidates for eyewear lens color, the step of selecting candidates for lens color of a contact lens or the step of selecting candidates for eyewear frame color, one to three colors or five or more colors may be determined for eyewear lens color and/or eyewear frame color based on the first color (first selected colors) and the second color (second selected colors).

Further, in a modification of the above-described embodiments, in the method for determining an eyewear lens color, the method for determining a lens color for a contact lens, and the method for determining an eyewear frame color, a lens color and/or a frame color may be determined based on the first color (first selected color) and the second color (second selected colors) only by an arithmetic expression without the aid of a table as described above.

Further, in a modification of the above-described embodiments, a lens color and a frame color may be simultaneously determined. In this case, for example, based on a table in which the first color (first selected colors) and the second color (second selected colors) are associated with lens color and frame color, a lens color and a frame color are simultaneously determined from the first color (first selected colors) and the second color (second selected colors).

Further, in a modification of the above-described embodiments, through a similar determination procedure to the determination procedure to determine a color of an eyewear lens, a contact lens or an eyewear frame, it is possible to determine also a color of any wearable thing worn by a person, other than an eyewear lens, a contact lens and an eyewear frame. A wearable thing as used herein may be preferably a wearable thing coming into direct contact with person's skin or a wearable thing to be visibly located when it is worn, but may be a wearable thing kept from direct contact with person's skin.

Further, in a modification of the above-described fourth embodiment, the processing terminal 5 may be used in the method for determining a lens color for a contact lens and the method for determining an eyewear frame color. For example, in the case of use in the method for determining an eyewear frame color, the processing terminal 5 is configured to be fit for use in the method for determining an eyewear frame color. For example, the frame color candidate selection table illustrated in FIG. 10 is stored as a frame color candidate selection database in the storage portion 30. And, on the output screen, a selection of one frame color may be made at the side of the determined four frame colors.

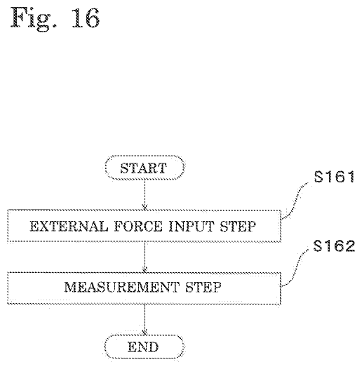

Further, as illustrated in FIG. 16, the above-described embodiments implement a trunk balance measurement method for measuring trunk balance of a person, which has an external force input step (step S161) and a measurement step (step S162), the external force input step (step S161) being of causing a person under evaluation of which trunk balance is to be measured to have readily an unstable posture upon external application of a force to the person under evaluation, then having the person under evaluation hold each of things of different colors in sequence, and externally applying a force to the person under evaluation, the measurement step (step S162) being of measuring trunk balance of the person under evaluation based on a movement of the person under evaluation in the external force input step.

Further, the above-escribed embodiments implement a human wearable thing color determination method for determining a color of a human wearable thing using the trunk balance measurement method, which has a color acquisition step and a color determination step, the color acquisition step being of acquiring color information on the thing providing measurement results of good trunk balance in the measurement step, the color determination step being of determining a color for the wearable thing of the person under evaluation based on the color information acquired in the color acquisition step.

Furthermore, in the color determination step, the method for determining a color of a human wearable thing is implemented in which a plurality of candidates for colors of the wearable thing is selected based on the color information acquired in the color acquisition step, and then the person under evaluation is caused to have readily an unstable posture upon external application of a force to the person under evaluation, who is then caused to wear the wearable thing with each of the plurality of candidate colors in sequence, in order to measure trunk balance of the person under evaluation based on a movement of the person under evaluation produced by externally applying a force to the person under evaluation, so that a color of the wearable thing that provides measurement results of good trunk balance is determined.

Also, while the embodiments of the present invention have been described, it should be apparent to those skilled in the art that changes can be made therein without departing from the sprit and scope of the invention. All such modifications and equivalents are intended to be included within the scope of the following claims.

### REFERENCE SIGNS LIST

1 plantar board
5 processing terminal
20 touch panel portion
30 storage portion
31 program
32 lens color candidate selection database
40 processing portion
41 color determination processing portion

1. A trunk balance measurement method for measuring trunk balance of a person, comprising:
an external force input step of causing a person under evaluation of which trunk balance is to be measured to have readily an unstable posture upon external application of a force to the person under evaluation, then causing the person under evaluation to hold each of things of different colors in sequence, and externally applying a force to the person under evaluation; and
a measurement step of measuring trunk balance of the person under evaluation based on a movement of the person under evaluation in the external force input step.

2. The trunk balance measurement method according to claim 1, wherein
in the external force input step, the person under evaluation is stood or sat on measuring means capable of causing the person under evaluation to have readily an unstable posture upon external application of a force to the person under evaluation.

3. The trunk balance measurement method according to claim 1, wherein
in the external force input step, the person under evaluation is stood on measuring means capable of causing the person under evaluation to stand to have readily an unstable posture upon external application of a force to the person under evaluation, and
the measuring means has a shape facilitating the person under evaluation having an unstable posture.

4. The trunk balance measurement method according to claim 3, wherein
the measuring means has a shape allowing a foot of the person under evaluation to be placed thereon, the shape extending from a heel to toes of the foot and having a smaller width than that of a sole of the foot, and
in the external force input step, the person under evaluation places one of his/her feet on the measuring means to stand on the one foot thereon, and then a force is externally applied to the person under evaluation.

5. The trunk balance measurement method according to claim 1, wherein
the things of different colors are colored paper sheets.

6. An eyewear lens color determination method for determining an eyewear lens color using the trunk balance measurement method according to claim 1, the eyewear lens color determination method comprising:
a color acquisition step of acquiring color information on the thing providing measurement results of good trunk balance in the measurement step; and
a color determination step of determining an eyewear lens color based on the color information acquired in the color acquisition step.

7. The eyewear lens color determination method according to claim 6, wherein,
in the color determination step, a plurality of candidates for eyewear lens colors is selected based on the color information acquired in the color acquisition step, then the person under evaluation is caused to have readily an unstable posture upon external application of a force to the person under evaluation, then the person under evaluation is caused to wear eyewear with each eyewear lens color of the plurality of candidates in sequence, in order to measure trunk balance of the person under evaluation based on a movement of the person under evaluation produced by externally applying a force to the person under evaluation, and an eyewear lens color that provides a measurement result of good trunk balance is determined.

8. A contact lens color determination method for determining a contact lens color using the trunk balance measurement method according to claim 1, the contact lens color determination method comprising:
a color acquisition step of acquiring color information on the thing providing measurement results of good trunk balance in the measurement step; and
a color determination step of determining a contact lens color based on the color information acquired in the color acquisition step.

9. The contact lens color determination method according to claim 8, wherein,
in the color determination step, a plurality of candidates for contact lens colors is selected based on the color information acquired in the color acquisition step, then the person under evaluation is caused to have readily an unstable posture upon external application of a force to the person under evaluation, then the person under evaluation is caused to wear either a contact lens or contact lenses with each color of the plurality of candidates in sequence, in order to measure trunk balance of the person under evaluation based on a movement of the person under evaluation produced by externally applying a force to the person under evaluation, and a contact lens color providing a measurement result of good trunk balance is determined.

10. An eyewear frame color determination method for determining an eyewear frame color using the trunk balance measurement method according to claim 1, the eyewear frame color determination method comprising:
a color acquisition step of acquiring color information on the thing providing measurement results of good trunk balance in the measurement step; and
a color determination step of determining an eyewear frame color based on the color information acquired in the color acquisition step.

11. The eyewear frame color determination method according to claim 10, wherein,
in the color determination step, a plurality of candidates for eyewear frame colors is selected based on the color information acquired in the color acquisition step, then the person under evaluation is caused to have readily an unstable posture upon external application of a force to the person under evaluation, then the person under evaluation is caused to wear eyewear with each eyewear frame color of the plurality of candidates in sequence, in order to measure trunk balance of the person under evaluation based on a movement of the person under evaluation produced by externally applying a force to the person under evaluation, and then an eyewear frame color providing a measurement result of good trunk balance is determined.

12. A human wearable thing color determination method for determining a color of a human wearable thing using the trunk balance measurement method according to claim 1, the wearable thing color determination method comprising:
a color acquisition step of acquiring color information on the thing providing measurement results of good trunk balance in the measurement step; and
a color determination step of determining a color of a wearable thing of the person under evaluation based on the color information acquired in the color acquisition step.

13. The human wearable thing color determination method according to claim 12, wherein,
in the color determination step, a plurality of candidates for colors of a wearable thing is selected based on the color information acquired in the color acquisition step, then the person under evaluation is caused to have readily an unstable posture upon external application of a force to the person under evaluation, then the person under evaluation is caused to wear a wearable thing with each color of the plurality of candidates in sequence, in order to measure trunk balance of the person under evaluation based on a movement of the person under evaluation produced by externally applying a force to the person under evaluation, and a color of the wearable thing providing a measurement result of good trunk balance is determined.
